(19) United States
(12) Patent Application Publication
(10) Pub. No.: US 2021/0261725 A1
(43) Pub. Date: Aug. 26, 2021

(54) DEGRADABLE MICROPARTICLE, DEGRADABLE PRODUCT COMPRISING THE SAME AND APPLICATION THEREOF

(71) Applicant: National Tsing Hua University, Hsinchu City (TW)

(72) Inventors: Jane Wang, Hsinchu City (TW); Pei-Hsuan Hung, Hsinchu City (TW)

(21) Appl. No.: 17/150,520

(22) Filed: Jan. 15, 2021

(30) Foreign Application Priority Data
Feb. 20, 2020 (TW) 109105520

(51) Int. Cl. C08G 63/127; A61K 8/85; A61Q 19/00; C08G 63/12

(52) U.S. Cl. CPC C08G 63/127; A61K 8/85; A61Q 19/00; C08G 2230/00; A61K 2800/10; A61K 2800/412; C08G 63/12

(57) ABSTRACT

Provided is a degradable microparticle with a grain size in a range of 2 micrometers to 1400 micrometers, and the degradable microparticle comprises poly(glycerol sebacate), poly(glycerol maleate), poly(glycerol succinate-co-maleate), poly(glycerol succinate), poly(glycerol malonate), poly(glycerol glutarate), poly(glycerol adipate), poly(glycerol pimelate), poly(glycerol suberate), poly(glycerol azelate), or any combination thereof. A degradable product produced from the degradable microparticles can obtain the desired degradation effect and can be produced by chemical synthesis to reduce the production cost. With these advantages, the applicability of the degradable microparticles is improved.

### CROSS-REFERENCE TO RELATED APPLICATION

Pursuant to 35 U.S.C. § 119(a), this application claims the benefit of the priority to Taiwan Patent Application No. 109105520 filed on Feb. 20, 2020. The content of the prior application is incorporated herein by its entirety.

### BACKGROUND OF THE INVENTION

### 1. Field of the Invention

The invention relates to a degradable material, especially to a degradable microparticle, a degradable product comprising the same and application thereof.

### 2. Description of Related Art

Plastic microbeads are granular materials with grain sizes in the micrometer range, which can be produced from polymer materials such as polyethylene (PE), polypropylene (PP), polystyrene (PS), polyethylene terephthalate (PET), poly(methyl methacrylate) (PMMA), and nylon, etc. The lubricity of liquid products such as toothpaste, shower gel, facial cleanser, scrubbing gel, etc., can be improved by using the ball bearing effect of the plastic microparticles.

However, pesticides, pollutants and environmental hormones are easily adsorbed to the plastic microparticles. These hardly-degraded plastic microparticles cause a lot of environmental pollutions, especially a large amount of marine waste will be produced, and even the plastic microparticles will be ingested by marine life, thereby endangering the entire ecological equilibrium. In view of these problems, several countries have begun to restrict the use of the plastic microparticles. In 2015, Cosmetics Europe—The personal Care Association recommended to several countries to discontinue the use of the plastic microparticles in cosmetics or personal care products by 2020. In the same year, the United States (U.S.) also passed the Microbead-Free Waters Act, which prohibited the use of the plastic microparticles in cosmetics in several stages. Since 2018, New Zealand also has banned the production and sale of personal care products containing plastic microparticles.

In view of these problems, several materials for the degradable microparticles have been developed. For example, U.S. patent publication No. 20140026916A1 provides a degradable microparticle, which comprises polyhydroxyalkanoate (PHA), and the degradable microparticle is added to cosmetics and personal care products such as toothpaste and exfoliating products to replace the use of the non-degradable plastic microparticles. In addition, U.S. patent publication No. 20150231042A1 provides a degradable microparticle made of poly(3-hydroxybutyrate-co-3-hydroxyvalerate) (PHBV), which is produced from hydroxybutyrate (HB) and hydroxyvalerate (HV), and the degradable microparticle is added to personal care products such as skin cleansing products to replace the use of the non-degradable plastic microparticles.

However, all of the aforesaid degradable materials such as PHA, HB and HV must be synthesized by bacteria, thereby causing excessive production cost of the degradable microparticles. As a result, there is still a need to provide other materials for the degradable microparticles to overcome the aforesaid problems of environmental pollutions and excessive production cost.

### SUMMARY OF THE INVENTION

In view of the above-mentioned drawbacks, one of the objectives of the present invention is to develop a degradable microparticle, which can be used to replace non-degradable plastic microparticles, thereby overcoming the environmental problems caused by the non-degradable plastic microparticles.

Another objective of the present invention is to solve the shortcoming that the materials of the degradable microparticles are synthesized by bacteria, resulting in high production cost of the degradable microparticles.

In order to achieve the above objectives, the present invention provides a degradable microparticle with a grain size in a range of 2 micrometers (μm) to 1400 μm, and a material of the degradable microparticle comprises poly(glycerol sebacate) (PGS), poly(glycerol maleate) (PGM), poly(glycerol succinate-co-maleate) (PGSMA), poly(glycerol succinate), poly(glycerol malonate), poly(glycerol glutarate), poly(glycerol adipate), poly(glycerol pimelate), poly(glycerol suberate), poly(glycerol azelate), or any combination thereof.

By using the aforesaid materials, the production cost of the degradable microparticles can be reduced and the environmental problems caused by the non-degradable plastic microparticles can be solved.

Preferably, the material of the degradable microparticle may comprise PGS and PGM. More preferably, the material of the degradable microparticle may comprise PGM.

Preferably, the mean of the grain size of the degradable microparticle may be in a range of 2 μm to 800 μm. More preferably, the mean of the grain size of the degradable microparticle may be in a range of 2 μm to 400 μm.

Preferably, a coefficient of variation (CV) of the grain size of the degradable microparticle may be in a range of 40% to 110%. More preferably, the CV of the grain size of the degradable microparticle may be in a range of 40% to 100%. Much more preferably, the CV of the grain size of the degradable microparticle may be in a range of 40% to 90%.

Preferably, a polydispersity index (PDI) of the grain size of the degradable microparticle may be in a range of 0.15 to 1.2. More preferably, the PDI of the grain size of the degradable microparticle may be in a range of 0.15 to 1.05. Much more preferably, the PDI of the grain size of the degradable microparticle may be in a range of 0.15 to 1.

According to the present invention, a shape of the degradable microparticle is not particularly limited. Preferably, the shape of the degradable microparticle may be spherical, water drop shaped, threaded, square, polyhedral, or any combination thereof.

Preferably, a structure of the degradable microparticle may be a solid structure, a hollow structure, a porous structure, or any combination thereof.

In order to achieve the objectives, the present invention provides a use of the degradable microparticle, which comprises preparing a degradable product from the degradable microparticle, and the degradable product can be a carrier in the process of drug delivery. In addition, the present invention provides a degradable product, which comprises the aforesaid degradable microparticle.

Preferably, the degradable product may be degraded in seawater or non-seawater. In an embodiment, a salinity of the seawater may be in a range of 32%0 to 38‰. Preferably, the salinity of the seawater may be in a range of 32%0 to 35‰.

Preferably, the degradable product may be degraded in an aqueous solution with a pH value greater than or equal to 4 and less than or equal to 10.

Preferably, the degradable product may be degraded in static water or flowing water. More preferably, the degradable product may be degraded in flowing water.

Preferably, the degradable product may be degraded in an enzyme solution. In an embodiment, a concentration of the enzyme solution may be in a range of 1 to 100 units per millimeter (units/mL).

### BRIEF DESCRIPTION OF THE DRAWINGS

FIG. 1A is a scanning electron microscope (SEM) image of a PGM microparticle of Example 10.

FIG. 1B is a particle-size distribution diagram of the PGM microparticle of Example 10.

FIGS. 2A to 2H are respectively SEM images of the PGM microparticle of Example 10 in a deionized water (DI water) observed on the 7^{th}, the 14^{th}, the 22^{th}, the 28^{th}, the 36^{th}, the 44^{th}, the 48^{th} and the 57^{th} days.

FIGS. 3A to 3C are respectively SEM images of the PGM microparticle of Example 10 in a buffer solution of pH 4 observed on the 2^{nd}, the 7^{th} and the 14^{th} days. FIGS. 3D to 3F are respectively SEM images of the PGM microparticle of Example 10 in a buffer solution of pH 6 observed on the 2^{nd}, the 7^{th} and the 14^{th} days. FIGS. 3G to 3I are respectively SEM images of the PGM microparticle of Example 10 in a buffer solution of pH 8 observed on the 2nd, the 7^{th} and the 14^{th} days. FIGS. 3J to 3L are respectively SEM images of a PGS microparticle of Example 14 in a buffer solution of pH 10 observed on the 0, the 8^{th} and the 28^{th} days. FIGS. 3M to 3O are respectively SEM images of a polylactic acid (PLA) microparticle of Comparative Example 1 in the buffer solution of pH 10 observed on the 0, the 8^{th} and the 28^{th} days. FIG. 3P is a diagram plotting pH curves of the DI water and buffer solutions when the PGM microparticle of Example 10 had been stored in them for a period of time. FIG. 3Q is a diagram plotting total organic carbon (TOC) curves of the buffer solutions when the PGM microparticle of Example 10 had been stored in them for a period of time. FIG. 3R is a diagram plotting TOC curve of the buffer solution of pH 10 when the PGM microparticle of Example 10 had been stored in it for a period of time. FIG. 3S is a diagram plotting TOC curves of the buffer solutions when the PLA microparticle of Comparative Example 1 had been stored in them for a period of time. FIG. 3T is a diagram plotting TOC curves of the buffer solution of pH 4 when the PGM microparticle of Example 10, the PGS microparticle of Example 14 and the PLA microparticle of Comparative Example 1 had been stored in it for a period of time. FIG. 3U is a diagram plotting TOC curves of the buffer solution of pH 6 when the PGM microparticle of Example 10, the PGS microparticle of Example 14 and the PLA microparticle of Comparative Example 1 had been stored in it for a period of time. FIG. 3V is a diagram plotting TOC curves of the buffer solution of pH 8 when the PGM microparticle of Example 10, the PGS microparticle of Example 14 and the PLA microparticle of Comparative Example 1 had been stored in it for a period of time. FIG. 3W is a diagram plotting TOC curves of the buffer solution of pH 10 when the PGM microparticle of Example 10, the PGS microparticle of Example 14 and the PLA microparticle of Comparative Example 1 had been stored in it for a period of time.

FIG. 4A is a diagram plotting TOC curves of the DI water and a synthetic seawater when the PGM microparticle of Example 10 had been stored in them for a period of time.

FIG. 4B is a diagram plotting TOC curves of the DI water and the synthetic seawater when the PLA microparticle of Comparative Example 1 had been stored in them for a period of time.

FIG. 5 is a diagram plotting TOC curves of static and flowing DI water and static and flowing synthetic seawater when the PGM microparticle of Example 10 had been stored in them for a period of time.

FIG. 6A is a SEM image of the PGM microparticle of Example 10 in a phosphate-buffered saline (PBS) containing enzyme observed on the 7^{th} day.

FIG. 6B is a SEM image of the PLA microparticle of Comparative Example 1 in the PBS containing enzyme observed on the 7^{th} day.

FIG. 6C is a diagram plotting amount of carboxylic acid curves of the PBS containing enzyme when the PGM microparticle of Example 10 and the PLA microparticle of Comparative Example 1 had been stored in it for a period of time.

### DETAILED DESCRIPTION OF THE PREFERRED EMBODIMENTS

Examples were made to prove the degradation effects of the degradable microparticles of the present invention. Comparative Example of plastic microparticle was made to compare with the Examples. One person skilled in the arts can easily realize the advantages and effects of the degradable microparticles in accordance with the present invention from the comparison of the Examples and the Comparative examples. The descriptions proposed herein are just for the purpose of illustrations only, not intended to limit the scope of the invention. Various modifications and variations could be made in order to practice or apply the present invention without departing from the spirit and scope of the invention.

### Examples 1 to 13: Preparation of PGM Microparticles

Firstly, glycerol and maleic acid (purchased from Sigma-Aldrich) were weighed at a mole ratio of 1:1, put into a two-neck bottle under an atmosphere of nitrogen gas and heated to a temperature of 130° C. for 0.5 hours to make the glycerol and the maleic acid fully dissolved and mixed, and then dehydrated under low pressure and a temperature of 160° C., so as to obtain a prepolymer. Finally, the prepolymer was cooled down to ambient temperature, and then was diluted with acetone with a purity of 99% at a weight ratio of 1:0.5 to 1:10, so as to obtain a prepolymer solution for use.

The aforesaid prepolymer solution was put into an injection pump, setting a caliber size of the injection pump in a range of 580 μm to 1200 μm, and then injecting the prepolymer solution at an injection rate of 0.1 milliliter per minute (mL/min) to 6.0 mL/min into a beaker containing silicon oil at a stirring speed of 400 revolutions per minute (rpm) to 1000 rpm at a temperature of 130° C. for 3 hours, so as to obtain a mixture. Subsequently, the mixture was filtered with membrane and washed with ethyl acetate to remove the unreacted silicone oil and/or the unreacted prepolymer, and then dried in a 50° C. oven for 24 hours, so as to obtain PGM microparticles of Examples 1 to 13.

The manufacturing parameters of the injection rate, the stirring speed, the dilution ratio and the caliber size corresponding to the PGM microparticle of each Example were listed in the following Table 1.

TABLE 1
the manufacturing parameters of the PGM microparticles
of Examples 1 to 13 (E1 to E13) and the PGS
microparticle of Example 14 (E14).

| Example | Injection rate | Stirring speed | Dilution ratio | Caliber size |
|---|---|---|---|---|
| No. | (mL/min) | (rpm) | (w/w) | (μm) |
| E1 | 0.1 | 1000 | 1:0.5 | 580 |
| E2 | 0.5 | 1000 | 1:0.5 | 580 |
| E3 | 1 | 1000 | 1:0.5 | 580 |
| E4 | 3 | 1000 | 1:0.5 | 580 |
| E5 | 6 | 1000 | 1:0.5 | 580 |
| E6 | 1 | 400 | 1:0.5 | 580 |
| E7 | 1 | 600 | 1:0.5 | 580 |
| E8 | 1 | 800 | 1:0.5 | 580 |
| E9 | 1 | 1000 | 1:1 | 580 |
| E10 | 1 | 1000 | 1:5 | 580 |
| E11 | 1 | 1000 | 1:10 | 580 |
| E12 | 1 | 1000 | 1:5 | 925 |
| E13 | 1 | 1000 | 1:5 | 1200 |
| E14 | 1 | 1000 | 1:10 | 580 |

The mean of the grain size, the standard deviation (SD) of the grain size, the CV of the grain size and the PDI of the grain size of the PGM microparticle of each Example were listed in the following Table 2. The SD, the CV and the PDI of the grain size all could be directed to the closeness of the grain size of each Example prepared by different manufacturing parameters; the lower the SD, the CV and the PDI represents that the grain size is more uniform.

The calculation method of the CV of the grain size (%): dividing the SD of the grain size by the mean of the grain size×100%. The calculation method of the PDI of the grain size: dividing the square of the SD of the grain size by the square of the mean of the grain size.

TABLE 2
the mean, the SD, the CV and the PDI of the grain size
of the PGM microparticles of Examples 1 to 13 (E1 to
E13) and the PGS microparticle of Example 14 (E14).

| Example | Mean | SD | CV | |
|---|---|---|---|---|
| No. | (μm) | (μm) | (%) | PDI |
| E1 | 29.9 | 25.4 | 84.9 | 0.72 |
| E2 | 31.2 | 26.4 | 84.9 | 0.72 |
| E3 | 39.4 | 23.7 | 60.3 | 0.36 |
| E4 | 79.5 | 80.5 | 101 | 1.03 |
| E5 | 89.1 | 89.9 | 99.2 | 0.98 |
| E6 | 112 | 77.7 | 64.9 | 0.48 |
| E7 | 101 | 81.3 | 79.9 | 0.64 |
| E8 | 66.8 | 41.9 | 62.7 | 0.39 |
| E9 | 60.0 | 26.1 | 43.4 | 0.19 |
| E10 | 30.2 | 13.0 | 43.1 | 0.19 |
| E11 | 26.3 | 15.7 | 59.8 | 0.36 |
| E12 | 39.7 | 20.1 | 50.6 | 0.26 |
| E13 | 32 | 19.1 | 59.7 | 0.36 |
| E14 | 26.0 | 13.6 | 52.3 | 0.27 |

### Examples 14: Preparation of PGS Microparticle

Firstly, glycerol and sebacic acid (purchased from Sigma-Aldrich) were weighed at a mole ratio of 1:1, put into a two-neck bottle under an atmosphere of nitrogen gas and heated to a temperature of 130° C. for 1 hour to make the glycerol and the sebacic acid fully dissolved and mixed, and then dehydrated under low pressure and a temperature of 130° C., so as to obtain a prepolymer. Finally, the prepolymer was cooled down to ambient temperature, and then was diluted with acetone with a purity of 99% at a weight ratio of 1:10, so as to obtain a prepolymer solution for use.

The aforesaid prepolymer solution was put into an injection pump, setting a caliber size of the injection pump of 580 μm, and then injecting the prepolymer solution at an injection rate of 1.0 mL/min into a beaker containing silicon oil at a stirring speed of 1000 rpm at a temperature of 160° C. for 5 hours, so as to obtain a mixture. Subsequently, the mixture was filtered with membrane and washed with ethyl acetate to remove the unreacted silicone oil and/or the unreacted prepolymer, and then dried in a 50° C. oven for 24 hours, so as to obtain the PGS microparticle of Example 14.

The manufacturing parameters of the PGS microparticle of Example 14 were listed in Table 1 above. The mean, the SD, the CV and the PDI of the grain size of the PGS microparticle of Example 14 were listed in Table 2 above.

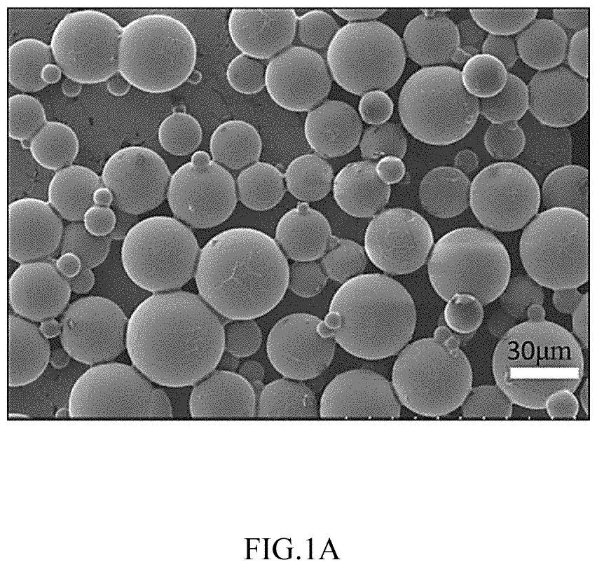

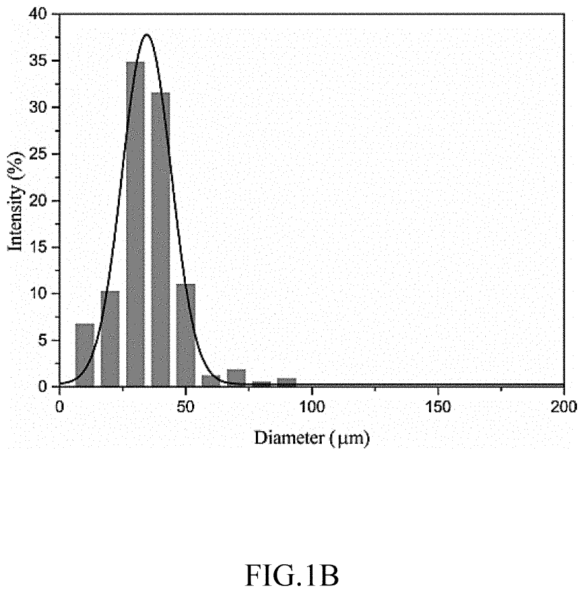

As shown in Table 2 above, the PDI of the grain size of the PGM microparticle of Example 10 was the lowest, which demonstrated that the grain size of the PGM microparticle of Example 10 was the most uniform compared with other examples. In addition, as shown in FIG. 1A and FIG. 1B, the PGM microparticle of Example 10 observed by SEM was spherical in shape and had a mean of the grain size of about 30 μm and an SD of the grain size of 13 μm. The aforesaid results demonstrated that the grain size of the PGM microparticle of Example 10 was the most uniform.

### Comparative Example 1: PLA Microparticle

The material of the PLA microparticle of Comparative Example 1 was purchased from Chiao Fu Material Technology Co., Ltd., and then was processed to obtain the PLA microparticle of Comparative Example 1.

### Test Example 1: DI Water

First, 250 milligrams (mg) of each of the PGM microparticle of Example 10, the PGS microparticle of Example 14 and the PLA microparticle of Comparative Example 1 was stored in 15 mL of DI water at a rotation speed of 175 rpm at ambient temperature for a period of time. After that, each degradable microparticle and the PLA microparticle were observed by SEM, so as to obtain the degradation results of them.

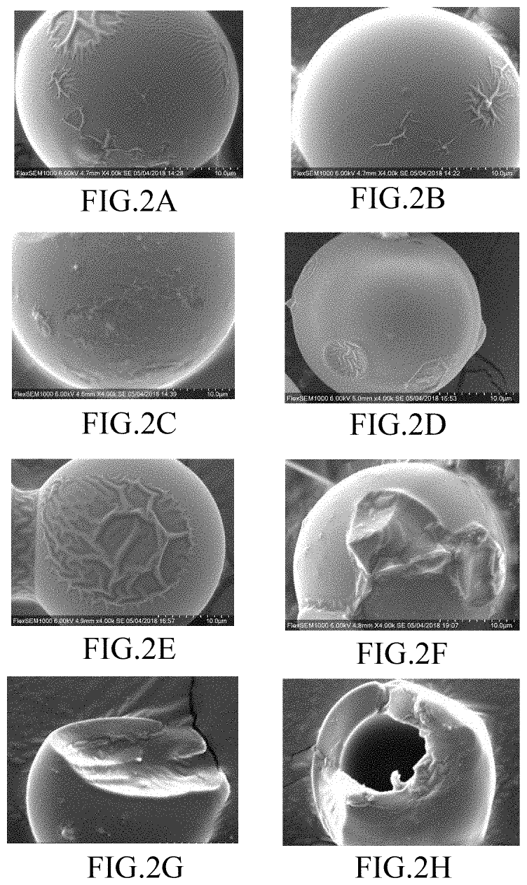

Take the degradation results of the PGM microparticle of Example 10 in the DI water for illustration, and the results are shown in FIGS. 2A to 2H. As shown in FIGS. 2A to 2D, the PGM microparticle of Example 10 in the DI water observed on the 28^{th} day was still complete with part of surface texture. As shown in FIGS. 2E to 2F, the PGM microparticle of Example 10 in the DI water observed on the 36^{th} day and the 44^{th} day could be observed some indents on the surface. As shown in FIGS. 2G to 2H, the PGM microparticle of Example 10 in the DI water observed on the 48^{th} day and the 57^{th} day no longer had the initial spherical shape, and the surfaces of the PGM microparticle of Example 10 had been cracked and the structure of the PGM microparticle of Example 10 had been broken. It demonstrated that the PGM microparticle of Example 10 could be degraded in the DI water.

### Test Example 2: DI Water and Buffer Solutions with Different pH

In this test example, the PGM microparticle of Example 10, the PGS microparticle of Example 14 and the PLA microparticle of Comparative Example 1 were stored in buffer solutions with different pH to test the degradation effect. First, 250 mg of each aforesaid microparticle was stored in 15 mL of buffer solutions with different pH at a rotation speed of 175 rpm at ambient temperature for a period of time. After that, each microparticle during different degradation processes was observed by SEM, and 20 μL of the buffer solutions during different degradation processes were taken to analyze the variation of the TOC of the buffer solutions by a TOC analyzer, so as to obtain the degradation results of the degradable microparticles and the PLA microparticle.

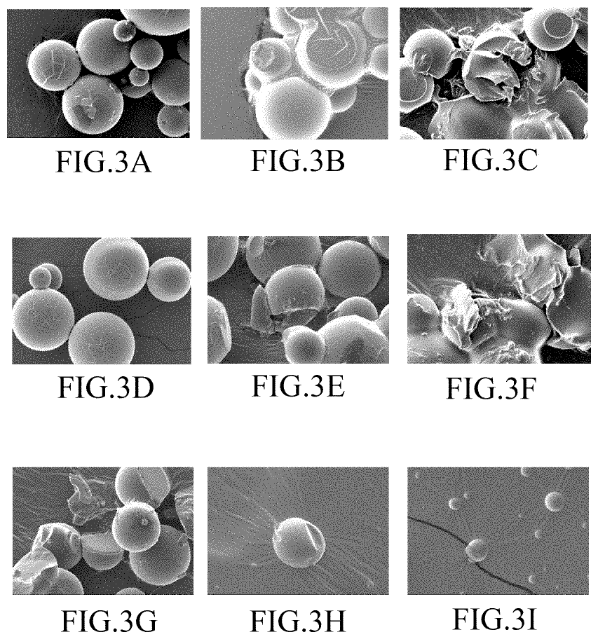

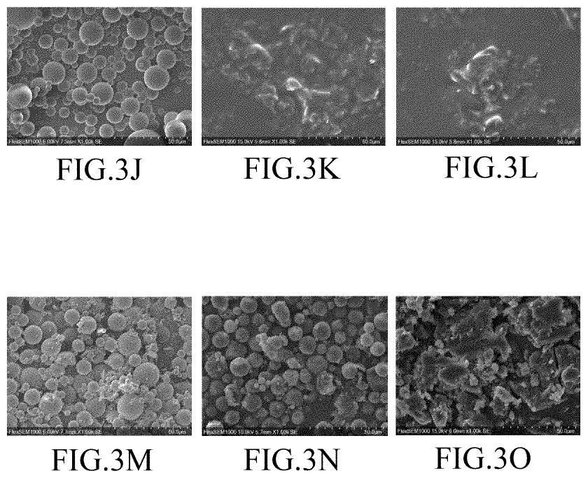

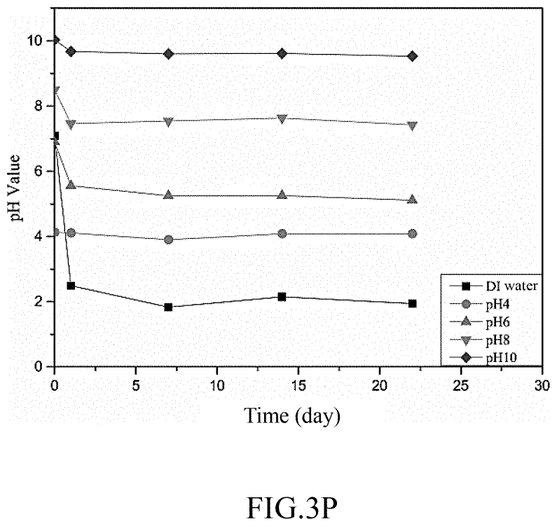

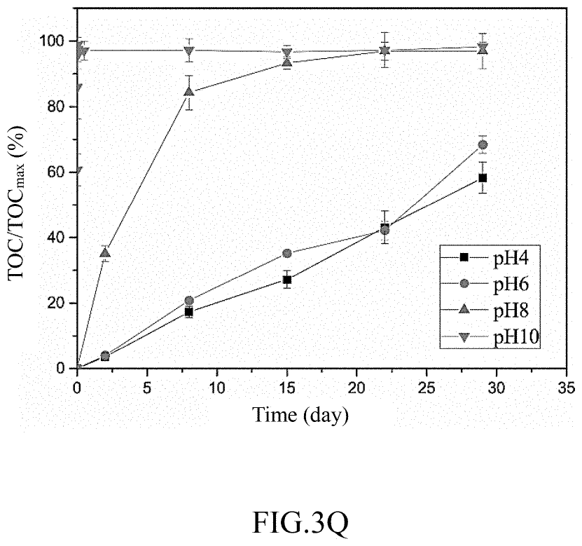

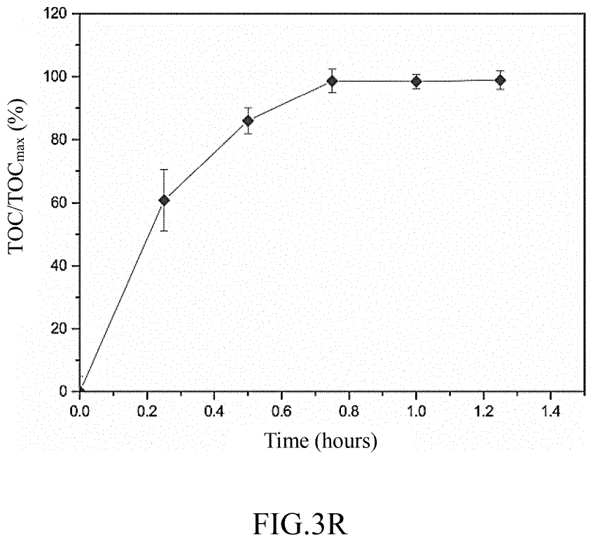

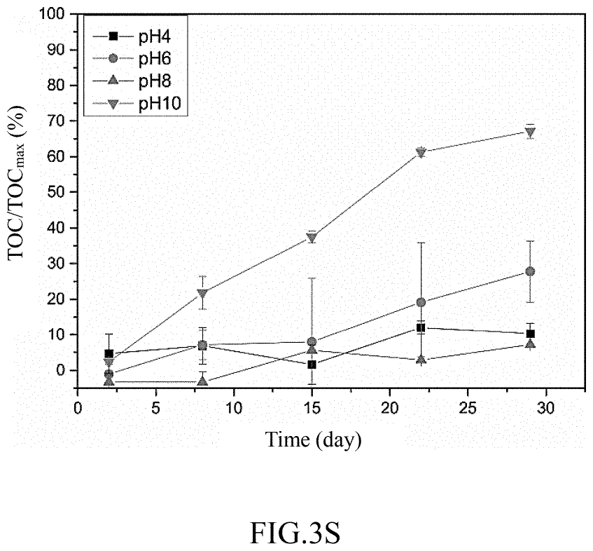

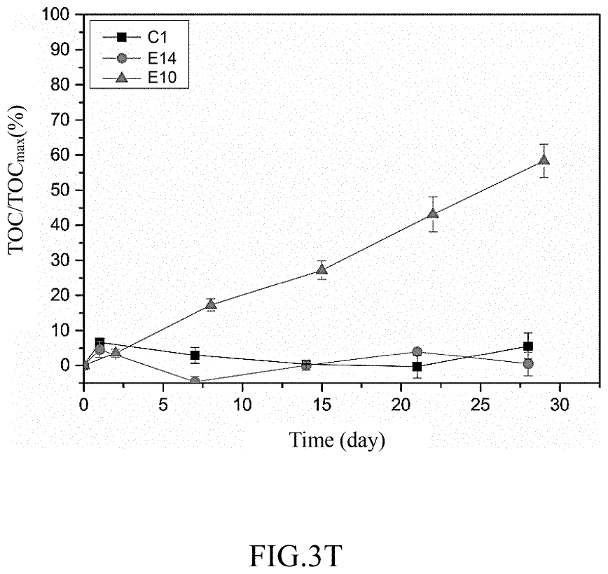

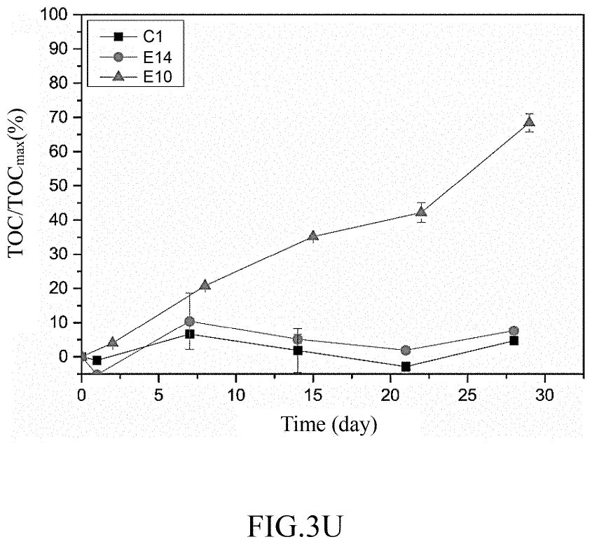

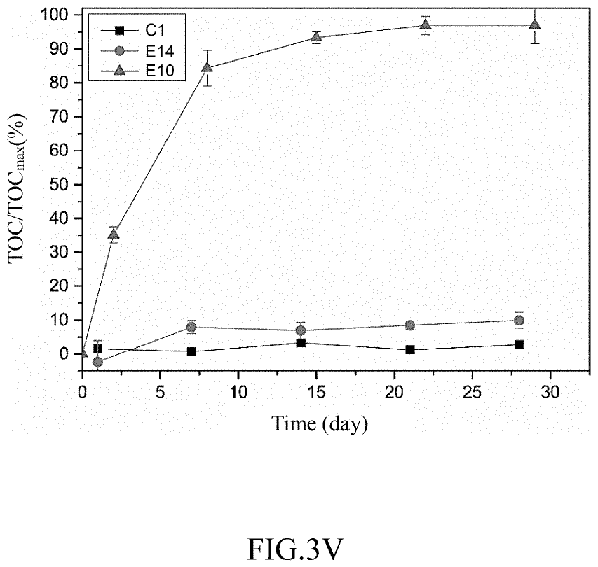

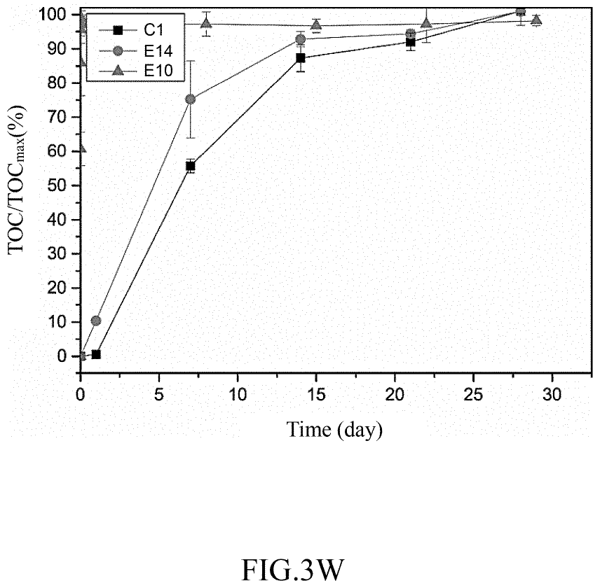

Take the degradation results of the PGM microparticle of Example 10 in the buffer solutions with different pH for illustration, and the results are shown in FIGS. 3A to 3I. As shown in FIGS. 3A to 3B, the PGM microparticle of Example 10 in a buffer solution of pH 4 on the 2^{nd} day and the 7^{th} day was still complete with part of surface texture. As shown in FIG. 3C, the PGM microparticle of Example 10 in the buffer solution of pH 4 on the 14^{th} day no longer had the initial spherical shape, and the surface of the PGM microparticle of Example 10 had been cracked and the structure of the PGM microparticle of Example 10 had been broken. Compared to FIGS. 3D to 3F and FIGS. 3G to 3I, the PGM microparticle of Example 10 respectively stored in buffer solutions of pH 6 and pH 8 could observe that the surface of the PGM microparticle of Example 10 had been cracked and the structure of the PGM microparticle of Example 10 had been broken with the storage time prolonged, even the PGM microparticle of Example 10 in the buffer solution of pH 8 on the 2nd day no longer had the initial spherical shape and the surface had been cracked and the structure had been broken. It demonstrated that the PGM microparticle of Example 10 could be degraded in the buffer solutions with different pH.

Take the degradation results of the PGS microparticle of Example 14 in a buffer solution of pH 10 for illustration, and the results are shown in FIGS. 3J to 3L. As shown in FIG. 3J, the PGS microparticle of Example 14 in the buffer solution of pH 10 on day 0 was still complete. As shown in FIGS. 3K to 3L, the PGS microparticle of Example 14 in the buffer solution of pH 10 on the 8^{th} day and the 28^{th} day no longer had the initial spherical shape, and the surface of the PGS microparticle of Example 14 had been cracked and the structure of the PGS microparticle of Example 14 had been broken. It demonstrated that the PGS microparticle of Example 14 could be degraded in the buffer solution of pH 10.

Take the degradation results of the PLA microparticle of Comparative Example 1 in the buffer solution of pH 10 for illustration, and the results are shown in FIGS. 3M to 3O. As shown in FIGS. 3M to 3N, the PLA microparticle of Comparative Example 1 in the buffer solution of pH 10 on 0 day and the 8th day were still complete. As shown in FIG. 3O, the PLA microparticle of Comparative Example 1 in the buffer solution of pH 10 on the 28^{th} day no longer had the initial spherical shape, and the surface of the PLA microparticle of Comparative Example 1 had been cracked and the structure of the PLA microparticle of Comparative Example 1 had been broken. It demonstrated that the PLA microparticle of Comparative Example 1 could be degraded in the buffer solution of pH 10.

Take the variation of pH of the DI water and the buffer solutions with different pH when the PGM microparticle of Example 10 had been stored in them for a period of time for illustration, and the results are shown in FIG. 3P. As shown in FIG. 3P, when the PGM microparticle of Example 10 was in the DI water, the buffer solutions of pH 4, pH 6, pH 8 and pH 10 during the period of time, the pH of the DI water and the buffer solutions both decreased significantly in a short time. It demonstrated that the PGM microparticle of Example 10 could be degraded in the DI water and the buffer solutions with different pH, and the maleic acid was released from the PGM microparticle of Example 10 during the degradation processes in the DI water and the buffer solutions, thereby causing the variation of pH of the DI water and the buffer solutions.

Next, take the TOC curves of the buffer solutions with different pH when the PGM microparticle of Example 10 had been stored in them for a period of time for illustration, and the results are shown in FIGS. 3Q to 3R. As shown in FIGS. 3Q to 3R, when the PGM microparticle of Example 10 had been in the buffer solutions of pH 4, pH 6, pH 8 and pH 10 for a period of time, the TOC of the buffer solutions all increased significantly with the storage time prolonged, and the increasing rate increased significantly with the increase of pH. It demonstrated that the PGM microparticle of Example 10 could be degraded in the buffer solutions with different pH, and its degradation speed increased significantly with the increase of pH.

As shown in FIG. 3S, when the PLA microparticle of Comparative Example 1 had been in the buffer solutions of pH 4, pH 6, pH 8 and pH 10 for a period of time, the TOC of the buffer solutions all increased significantly with the storage time prolonged. It demonstrated that the PLA microparticle of Comparative Example 1 could be degraded in the buffer solutions with different pH.

As shown in FIGS. 3T to 3V, when the PGM microparticle of Example 10, the PGS microparticle of Example 14 and the PLA microparticle of Comparative Example 1 had been stored in the buffer solutions of pH 4, pH 6 and pH 8 for a period of time, the degradation effect of the PGM microparticle of Example 10 was better than that of the PGS microparticle of Example 14 and the PLA microparticle of Comparative Example 1. In addition, comparing the results of the degradation effects of the PGS microparticle of Example 14 and the PLA microparticle of Comparative Example 1, the degradation effect of the PGS microparticle of Example 14 was also better than that of the PLA microparticle of Comparative Example 1.

As shown in FIG. 3W, when the PGM microparticle of Example 10, the PGS microparticle of Example 14 and the PLA microparticle of Comparative Example 1 had been stored in the buffer solution of pH 10 for a period of time, the TOC of the buffer solutions all increased significantly with the storage time prolonged. It demonstrated that the PGM microparticle of Example 10, the PGS microparticle of Example 14 and the PLA microparticle of Comparative Example 1 could be degraded in the buffer solution of pH 10.

### Test Example 3: DI Water and Synthetic Seawater

In this test example, the PGM microparticle of Example 10 and the PLA microparticle of Comparative Example 1 were stored in different types of water to test the degradation effect. First, 250 mg of each aforesaid microparticle was stored in 15 mL of the DI water and 15 mL of synthetic seawater at a rotation speed of 175 rpm at ambient temperature for a period of time. After that, 20 μL of the DI water and 20 μL of the synthetic seawater at different times during degradation were taken to analyze the variation of the TOC of the DI water and the synthetic seawater by the TOC analyzer, so as to obtain the degradation results of the degradable microparticle and the PLA microparticle.

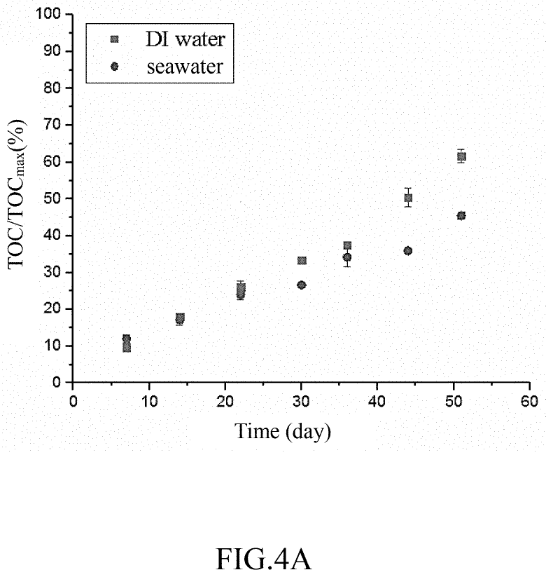

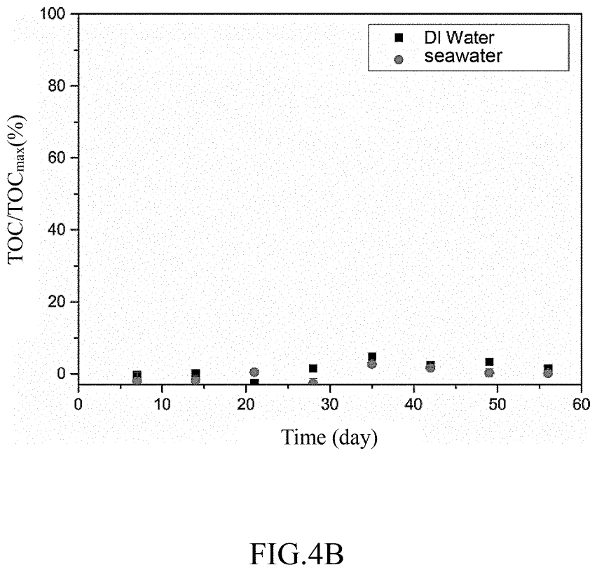

As shown in FIG. 4A, when the PGM microparticle of Example 10 had been in the DI water and the synthetic seawater for a period of time, the TOC of the DI water and the TOC of the synthetic seawater both increased significantly with the storage time prolonged. It demonstrated that the PGM microparticle of Example 10 could be degraded in different water types.

As shown in FIG. 4B, when the PLA microparticle of Comparative Example 1 had been in the DI water and the synthetic seawater for a period of time, the TOC of the DI water and the TOC of the synthetic seawater both remained unchanged with the storage time prolonged. It demonstrated that the PLA microparticle of Comparative Example 1 could not have an ideal degradation effect in different water types.

### Test Example 4: Static and Flowing DI Water and Static and Flowing Synthetic Seawater

In this test example, the PGM microparticle of Example 10 was stored in water of different fluidities to test the degradation effect. First, 250 mg of each aforesaid microparticle was stored in 15 mL of the DI water and 15 mL of the synthetic seawater at a rotation speed of 175 rpm at ambient temperature for a period of time. Both of the DI water and the synthetic seawater were divided into two groups: one of the groups had the water renewed every two days as flowing water, and the other group had the water unchanged as static water for comparison. After that, 20 μL of the DI water and 20 μL of the synthetic seawater at different times during degradation were taken to analyze the variation of the TOC of the DI water and the synthetic seawater by the TOC analyzer, so as to obtain the degradation results of the degradable microparticle.

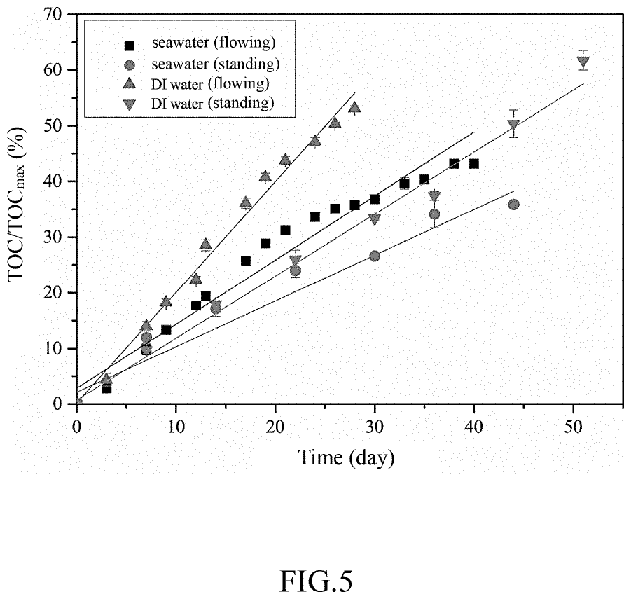

As shown in FIG. 5, when the PGM microparticle of Example 10 had been in the DI water and the synthetic seawater for a period of time, the TOC of the DI water and the TOC of the synthetic seawater both increased significantly with the storage time prolonged, and particularly, the TOC of the water-renewed group increased much higher than that of the water-unchanged group. It demonstrated that the PGM microparticle of Example 10 could be degraded in water of different fluidities, especially in flowing water.

### Test Example 5: Enzyme Solution

In this test example, the PGM microparticle of Example 10 and the PLA microparticle of Comparative Example 1 were stored in 20 units/mL of an enzyme solution to test the degradation effect. First, 250 mg of each aforesaid microparticle was stored in 15 mL of a PBS containing 10 units/mL of lipase of pH 7.4 at a rotation speed of 175 rpm at ambient temperature for a period of time. After that, each microparticle was observed by SEM at different times, and 20 μL of the PBS at different times during degradation was taken to analyze the variation of the carboxylic acid of the PBS by an ultraviolet-visible spectrophotometer, so as to obtain the degradation results of the degradable microparticle and the PLA microparticle.

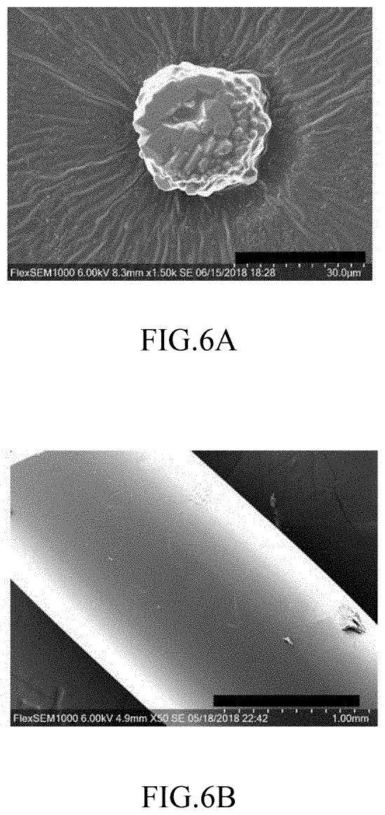

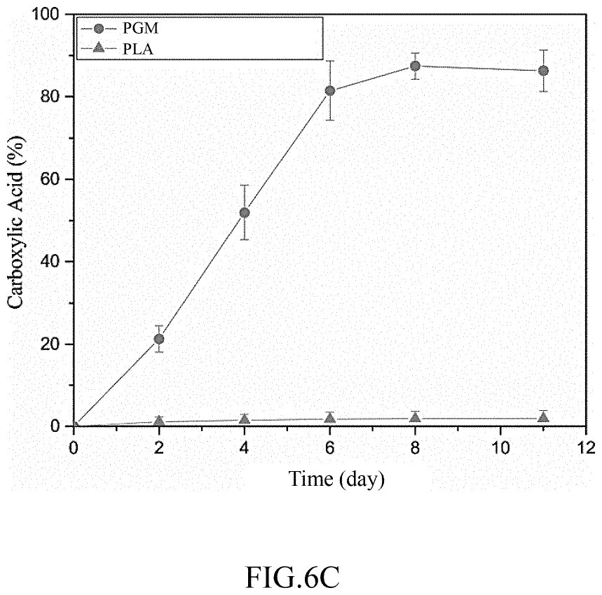

Take the degradation result of the PGM microparticle of Example 10 in the PBS containing enzyme for illustration, and the result is shown in FIG. 6A. As shown in FIG. 6A, the PGM microparticle of Example 10 in the PBS containing lipase observed on the 7^{th} day no longer had the initial spherical shape, and the surface of the PGM microparticle of Example 10 had been cracked and the structure of the PGM microparticle of Example 10 had been broken. It demonstrated that the PGM microparticle of Example 10 could be degraded in the PBS containing enzyme.

Take the degradation result of the PLA microparticle of Comparative Example 1 in the PBS containing enzyme for illustration, and the result is shown in FIG. 6B. As shown in FIG. 6B, the PLA microparticle of Comparative Example 1 in the PBS containing lipase observed on the 7^{th} day was still complete. It demonstrated that the PLA microparticle of Comparative Example 1 had not been degraded at all on the 7^{th} day in the PBS containing enzyme.

As shown in FIG. 6C, the degradation effect of the PGM microparticle of Example 10 in the PBS containing lipase was better than that of the PLA microparticle of Comparative Example 1 in the PBS containing lipase.

According to the results of Test Examples 1 to 5, the degradable microparticles of the present invention can obtain the desired degradation effect under different conditions such as solutions with different pH values, water types, fluidities, and solutions containing enzyme. In addition, the degradable microparticles of the present invention can be produced by chemical synthesis to reduce the production cost. With aforesaid advantages, the technical means of the present invention further improves the applicability of the degradable microparticles and replaces the use of the plastic microparticles, thereby overcoming the environmental problems caused by the plastic microparticles.

Even though numerous characteristics and advantages of the instant disclosure have been set forth in the foregoing description, together with details of the structure and features of the disclosure, the disclosure is illustrative only. Changes may be made in the details, especially in matters of material, shape, size, and arrangement of parts within the principles of the disclosure to the full extent indicated by the broad general meaning of the terms in which the appended claims are expressed.

What is claimed is:

1. A degradable microparticle, wherein a material of the degradable microparticle comprises poly(glycerol sebacate), poly(glycerol maleate), poly(glycerol succinate-co-maleate), poly(glycerol succinate), poly(glycerol malonate), poly(glycerol glutarate), poly(glycerol adipate), poly(glycerol pimelate), poly(glycerol suberate), poly(glycerol azelate), or any combination thereof; a grain size of the degradable microparticle is in a range of 2 micrometers to 1400 micrometers.

2. The degradable microparticle as claimed in claim 1, wherein a polydispersity index of the grain size of the degradable microparticle is in a range of 0.15 to 1.2.

3. The degradable microparticle as claimed in claim 1, wherein a shape of the degradable microparticle is spherical, water drop shaped, threaded, square, polyhedral, or any combination thereof.

4. The degradable microparticle as claimed in claim 1, wherein a structure of the degradable microparticle is a solid structure, a hollow structure, a porous structure, or any combination thereof.

5. A use of the degradable microparticle as claimed in claim 1, which comprises preparing a degradable product from the degradable microparticle.

6. The use of the degradable microparticle as claimed in claim 5, wherein a polydispersity index of the grain size of the degradable microparticle is in a range of 0.15 to 1.2.

7. The use of the degradable microparticle as claimed in claim 5, wherein a shape of the degradable microparticle is spherical, water drop shaped, threaded, square, polyhedral, or any combination thereof.

8. The use of the degradable microparticle as claimed in claim 5, wherein a structure of the degradable microparticle is a solid structure, a hollow structure, a porous structure, or any combination thereof.

9. The use as claimed in claim 5, wherein the degradable product is able to be degraded in seawater or non-seawater.

10. The use as claimed in claim 5, wherein the degradable product is able to be degraded in an aqueous solution with a pH value greater than or equal to 4 and less than or equal to 10.

11. The use as claimed in claim 5, wherein the degradable product is able to be degraded in static water or flowing water.

12. The use as claimed in claim 5, wherein the degradable product is able to be degraded in an enzyme solution.

13. A degradable product, comprising the degradable microparticle as claimed in claim 1.

14. A degradable product, comprising the degradable microparticle as claimed in claim 2.

15. A degradable product, comprising the degradable microparticle as claimed in claim 3.

16. A degradable product, comprising the degradable microparticle as claimed in claim 4.
